(19) United States
(12) Patent Application Publication
(10) Pub. No.: US 2003/0134815 A1
(43) Pub. Date: Jul. 17, 2003

(54) Adenovirus mediated transfer of genes to the gastrointestinal tract

(75) Inventor: Ronald G. Crystal, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: The Govt. of the U.S.A., as Represented by the Secretary, Department of Health & Human Services, Rockville, MD

(21) Appl. No.: 10/300,209

(22) Filed: Nov. 20, 2002

Related U.S. Application Data
(63) Continuation of application No. 07/776,057, filed on Oct. 16, 1991, now abandoned.
Application No. 07/776,057 is a continuation-in-part of application No. 07/747,371, filed on Aug. 20, 1991, now abandoned.

(51) Int. Cl. A61K 48/00; C12N 15/861

(52) U.S. Cl. 514/44; 424/93.2; 435/456

(57) ABSTRACT

The present invention relates, in general, to a adenovirus mediated transfer of genes to the gastrointestinal tract. In particular, the present invention relates to a method of recombinant, replication-deficient adenovirus mediated transfer of therapuetic genes to the gastrointestinal tract whereby therapeutic proteins for systemic and/or local purposes are produced.

### BACKGROUND OF THE INVENTION

This application is a continuation-in-part of U.S. application Ser. No. 07/747,371 filed Aug. 20, 1991 the contents of which are incorporated herein by reference.

### FIELD OF THE INVENTION

The present invention relates, in general, to a method of adenovirus mediated transfer of genes to the gastrointestinal tract. In particular, the present invention relates to a method of recombinant, replication-deficient adenovirus mediated transfer of therapeutic genes to the gastrointestinal tract for the purpose of producing therapeutic proteins for systemic and/or local use.

### BACKGROUND INFORMATION

The use of proteins as therapeutic agents is limited inter alia by the physiologic barrier of the gastrointestinal tract. The terms protein, polypeptide, peptide, or segment of amino acids are herein used interchangeably to define a polymer of amino acids linked through peptide bonds. Therapeutic proteins are defined herein as proteins advantageous to an individual. Proteins cannot be administered for therapeutic purposes by the oral or rectal routes because they will not generally reach the circulation in an intact form in concentrations needed for therapy (the proteins are degraded and/or not absorbed). Consequently, therapeutic proteins need to be administered systemically for example, by the intravenous, subcutaneous, intradermal or intramuscular routes.

This problem of the administration has been dramatically heightened by the development of recombinant DNA technology, where it is possible to produce many different therapeutic proteins, all of which have to be administered systemically. While this may not be a major problem for short term use, long term use (which is the typical use for most of the recombinant proteins) requires long term systemic administration with all of the attendant problems with access route (e.g., veins available, discomfort and cost).

It is known that recombinant adenoviruses can be used to produce human protein in vivo (examples include injection of recombinant adenovirus intravenously into the portal vein to the liver, and intratracheal to the lung. All publications mentioned herein are hereby incorporated in their entirety by reference. (see Rosenfeld M et al. (1991) Science 252:431-434; Jaffe H A et al. (1991) Clin Res 39(2) 302A; Rosenfeld M A et al. (1991) Clin Res 39(2): 311A). However, all of these approaches are impractical to use for systemic administration of recombinant proteins because they require parenteral administration of the recombinant gene (i.e., intravenous, intraportal, intratracheal).

The present invention circumvents this by providing a method of administering therapeutic proteins by enteral routes by using a recombinant, replication deficient adenovirus containing the coding sequences of the gene of the therapeutic protein to insert the gene into the lining cells of the gastrointestinal tract, and using that site to produce the protein and secrete it into the circulation where the therapeutic protein would be available for systemic use. As an alternative, the same approach can be used to secrete proteins into the gastrointestinal tract for local therapeutic use within the lumen of the gastrointestinal tract, or for use within the cells or extracellular matrix of the walls of the gastrointestinal tract.

Studies in the 1960's demonstrated that live adenovirus placed into enteric coated capsules (to avoid inactivation in the stomach) and administered to humans by the oral route resulted in systemic immunization against the adenovirus (Chanock R M et al. (1966) JAMA 195:151-158). This is now a standard immunization procedure against adenovirus for military recruits in the USA. The concept underlying this immunization strategy is that the adenovirus will leave the capsule as it dissolves in the lumen of the intestine, infect the intestinal epithelial cells, replicate in the epithelial cells and the resulting shed newly replicated virus presents itself to the immune system, resulting in systemic immunity against the adenovirus.

Previously, it has been demonstrated that the adenovirus can be modified so that it is replication deficient (i.e., will not direct the production of new virus after it infects its target cell) and so that it contains new genes (e.g., the coding sequences of human genes of therapeutic interest). The use of recombinant DNA inserts under the direct control of the early promoter (EP) of the E1A region of the adenonvirus genome has been described by M. Perricaudet, et al. of the Pasteur Institute in European Patent Application No. 0185573, published Jun. 25, 1986. Such a modified virus can be used to transfer the recombinant gene to target cells in vivo (for examples, see Rosenfeld M et al. (1991) Science 252:431-434; Berkner K L (1988) BioTechniques 6:616-629).

### SUMMARY OF THE INVENTION

It is a general object of this invention to provide a method of producing a protein in the cells of the gastrointestinal tract of a patient.

It is a specific object of this invention to provide a method of producing a protein in the cells of the gastrointestinal tract of a patient. The method comprises administering to the patient's gastrointestinal tract a replication deficient adenovirus comprising a DNA segment encoding the protein under conditions such that the protein is produced. Depending on the specific sequences placed into the recombinant adenovirus, the protein would preferably then be secreted for systemic therapy to the circulation, for local therapy to the lumen of the gastro intestinal tract, or both. Further, the design of the recombinant adenovirus may preferably be to deliver the protein for use within the cells of the gastrointestinal tract or in the walls of the gastrointestinal tract.

Further objects and advantages of the present invention will be clear from the description that follows.

### BRIEF DESCRIPTION OF THE DRAWINGS

FIG. 1. Recombinant adenovirus (Ad) vector. Top—wild type Ad5 genome showing the E1a, E1b [map units (mu) 1.3-11.2; 100 mu=36 kb] and E3 (mu 76.6-86.0) regions.

FIG. 2. Anatomy of the colon wall and the cultured epithelial cell model used to evaluate the polarity of secretion of α1-antitrypsin produced by T84 human colon carcinoma epithelial cells modified with the recombinant adenovirus Ad-α1AT (ATCC CCL 248).

A. Cross-section of the colon wall showing the epithelial cells, the lumen of the colon, the apical surface of the epithelium abutting the lumen, the basolateral surface of the epithelium abutting the submucosa and thus the capillaries, and the muscle layer.

B. Chamber for epithelial cell cultures showing the microporous membrane, the cultured cells, and the separated apical and basolateral compartments.

FIG. 3. Demonstration of de novo synthesis and secretion of human α1-antitrypsin (α1AT) by rat colon exposed to the recombinant adenovirus Ad-α1AT ex vivo.

### DETAILED DESCRIPTION OF THE INVENTION

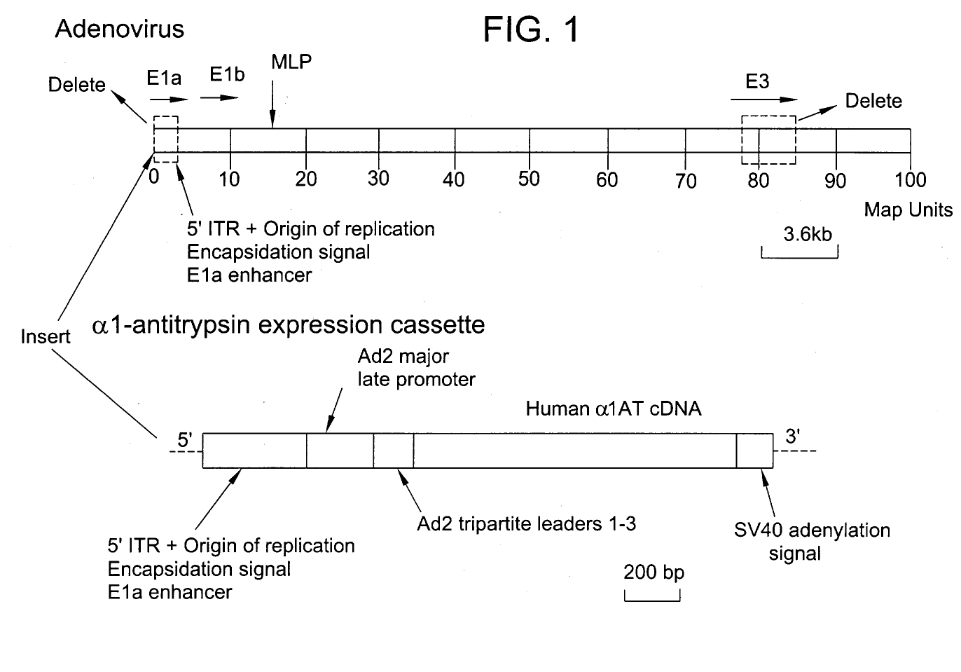

The present invention relates to a method of producing therapeutic proteins in the gastrointestinal tract. As a step, first, a replication deficient adenovirus (referred to below as the “modified adenovirus”) is constructed with the coding sequences of the protein of therapeutic interest. As an example, the adenovirus Ad-α1AT containing the coding sequence of the human α1AT gene is used (see FIG. 1 and Rosenfeld M et al. (1991) Science 252:431-434). Second, the modified adenovirus is placed into an enteric capsule (or alternatively, administered via tube past the stomach, orally or by tube into the stomach after the stomach lining fluid has been modified such that the virus will not be altered; or via the rectal route). Alternatively, a special coating may be applied to the adenovirus to prevent release and absorption of the modified adenovirus until the tablet reaches the basic (pH) environment of the duodenum, jejunum, ileum, or colon. For oral administration a capsule tablet or pill are convienent delivery vehicles; for rectal administration, a suppository may be preferred. Administration is then effected. The following scenario then occurs: (1) the cells (preferably, the epithelial cells) of the gastrointestinal tract are infected by the modified adenovirus; (2) the recombinant gene in the modified adenovirus sequence directs the synthesis of the recombinant protein which (depending upon how the sequences in the recombinant gene are engineered) can then be secreted into the circulation, into the lumen of the gastrointestinal tract or into both the circulation and the lumen of the gastrointestinal tract or within the epithelial cells of the gastrointestinal tract or in the local environs within the wall of the gastrointestinal tract; and (3) the therapeutic protein is then available to act systemically (when secreted into the circulation) or in the intestine (when secreted into the lumen), within the cells and/or the extracellular matrix of the wall of the gastrointestinal tract.

The present invention provides a practical, easy and safe way to administer recombinant proteins to humans. By using a replication deficient adenovirus, the process is safe because the virus cannot replicate in the target cells. By using a recombinant adenovirus, the target cells will produce the human therapeutic protein. By choosing the epithelium of the gastrointestinal tract for the target of infection by the replication deficient recombinant adenovirus, the invention permits ease of administration (preferably, by oral route via enteric coated capsule) via a route that can be used repetitively (for example, daily, or less frequently, depending on the chronicity of the recombinant adenovirus infection in the epithelial cells) and safely.

Because the epithelial cells of the gastrointestinal tract will secrete some of the product of the recombinant adenovirus through the basolateral surface of the infected epithelial cells, the method is available for applications requiring systemic use. Because these epithelial cells also secrete some of the product through their apical surface, the method is available for applications requiring luminal use (for example, intraluminal gastrointestinal disorders and gastrointestinal cancer). If the recombinant adenovirus is designed appropriately, the therapeutic protein will be available for therapeutic use within the epithelial cells of the gastrointestinal tract or in the local environs of the wall of the gastrointestinal tract (for example, for gastrointestinal tract cancer or gastrointestinal inflammatory disorders).

For disorders requiring systemic administration, this approach provides an easy and safe manner of administering recombinant protein to the circulation. Examples of such proteins include, but are not limited to:

α1-antitrypsin—for α1-antitrypsin deficiency

factor VIII—for hemophilia

other coagulation factors—for bleeding disorders

growth hormone—for growth disorders

insulin—for diabetes

other peptide hormones

other pituitary hormones [adrenal cortical stimulating hormone (ACTH) and thyroid stimulating hormone (TSH) are just two examples]

other lymphokines and cytokines for systemic therapy

interferon γ—for granulomatous disease of childhood (and other diseases being investigated)

interferon α—for leukemia and chronic active hepatitis

erythropoietin—for chronic renal failure and other marrow suppressive disorders

other hematologic growth factors—for marrow suppressive disorders

administration e.g., tissue plasminogen activator for prevention of thrombosis in the pulmonary coronary arteries following reperfusion therapy, especially after balloon catheterization, or CD4 for human immunodeficiency virus (HIV) infection, and other recombinant proteins requiring systemic administration, whether short term or long term

recombinant proteins for other hereditary disorders such as cerebrosidase deficiency and adenosine deaminase deficiency

receptor agonists or antagonists—for example, for the control of systemic hypertension; interleukin-1 receptor antagonist for septic shock, rheumatoid arthritis and other disorders

binding proteins for cytokines, lymphokines, and hormones—for example, tumor necrosis factor binding protein (a portion of the tumor necrosis factor receptor) for the treatment of shock and wasting disorders mediated by tumor necrosis factor

For hereditary and acquired disorders of the gastrointestinal tract, this approach provides a means of administering recombinant proteins to the surface or within the cells or extracellular matrix of the walls of the gastrointestinal tract. Examples of possible applications include:

pancreatic enzymes for pancreatic deficiency disorders such as cystic fibrosis

lactase for lactose intolerance and the appropriate enzymes for the small intestine disaccharidase deficiencies

local therapy for gastrointestinal cancers with cytokines, tumor suppressor proteins (for example, p53 and retinoblastoma genes), and cytotoxic proteins

prevention of cancer in individuals prone to gastrointestinal tract cancer (e.g., familial polyposis) with tumor suppressor proteins (for example, p53 and retinoblastoma genes).

For mammals and birds (more specifically, farm animals, for example—pigs, cattle, sheep, horses, dogs, cats, and chickens), this approach provides a means of administering recombinant proteins (for example, growth hormone) to these animals for the purposes of augmenting growth, generating characteristics for commercial purposes, and/or for general therapeutic purposes and for producing proteins from purified fractions given to humans, as well as antibodies for reagents reactive with human protein.

The use of proteins and polypeptides as therapeutic agents is greatly expanded according to the present invention by providing a means for delivering effective amounts of biologically active protein to a recipient individual. The preparations of this invention are suitably administered to animals, which include but are not limited to mammals (including humans), fish, and avians. The preparations are preferably administered to livestock (including cattle, horses, swine, sheep, goats, etc.), household pets (cats, dogs, canaries, parakeets, etc.) fish (especially in an aquarium or acquaculture environment, e.g., tropical fish, goldfish and other ornamental carp, catfish, trout, salmon, etc.) and avians, especially poultry such as chickens, ducks, geese, etc.

In one embodiment of the present invention, the replication-deficient adenovirus can be employed with animal feeds (or, with less dosage control, with animal drinking water) acting as a nontoxic, pharmaceutically acceptable carrier for administration to animals, e.g., livestock, household pets, fish, poultry, etc. In one aspect, this embodiment is useful for producing proteins for purification to human sera and to produce antibodies for reagents against human proteins in a different species, e.g., cattle, horse, sheep, goat, rabbit, swine, etc. In another aspect, this embodiment is useful in the treatment of disorders in which proteins or polypeptides are useful therapeutic agents, particularly when the gene coding the therapeutic protein is derived from the species being treated, or with sequences which are closely homologous to prevent immune reactions.

The replication-deficient adenovirus of the present invention can likewise be employed in admixture with conventional excipients, i.e., pharmaceutically acceptable organic or inorganic carrier substances suitable for enteral (e.g., oral) application that do not deleteriously react with the virus. Suitable pharmaceutically acceptable carriers are well known in the art. (Suitable vehicles include those that are acid resistant and base sensitive, that is, sufficiently so such that transport can be effected through the stomach without unacceptable degradation.) They include but are not limited to water, salt solutions, alcohols, gum arabic, vegetable oils, benzyl alcohols, polyethylene glycols, gelatine, carbohydrates such as lactose, anylose or starch, magnesium stearate, talc, silicic acid, viscous paraffin, perfume oil, fatty acid, monoglycerides and diglycerides, pentacrythritol fatty acid esters, hydroxy methyl cellulose, polyvinyl pyrrolidone, etc. Taking appropriate precautions not to kill the replication-deficient adenovirus, the preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active virus. For example, agents can be used to increase the pH of the stomach, either directly (such as by buffers or bases) or indirectly (such as by drugs), to allow the virus to more readily pass through unharmed. They also can be combined where desired with other biologically active agents, e.g., antisense DNA or mRNA.

In another embodiment of the present invention, the replication-deficient adenovirus can be employed as a vaccine to develop immunity against infectious agents. The strategy is as follows. The gene coding for the protein against which immunity is to be developed is cloned into the replication deficient adenovirus construct; next, the replication deficient adenovirus containing the gene of interest is then admininistered to an animal (preferably, a human) as described herein. The gene sequences are designed such that the protein is secreted by the epithelial cells of the gastrointestinal tract into the systemic circulation such that immunity against the foreign proteins is developed.

Examples of this approach include the development of immunity against the hepatitis viruses, human immunodeficiency virus, and all other viruses that cause animal disease (particularly human disease). This strategy may also be used to develop immunity against bacteria, fungi, and other infectious agents.

A particularly interesting aspect of the present invention involves the use of replication-deficient adenovirus as a delivery system for chemotherapeutic agents, including antisense compounds, especially for use in cancer chemotherapy. Use with conventional chemotherapeutic agents is as discussed above. Briefly, use with antisense compounds for use against tumor cells in the bowel involves selecting mRNA as the primary drug target, with either another mRNA molecule or a synthetic oligo deoxynucleotide having the complementary base sequence to the mRNA forming a hybrid duplex by hydrogen-bonded base pairing. This hybridization can prevent expression of the target mRNA's protein product, a process called “translation arrest”. Inhibition of mRNA is more efficient than inhibition of an enzyme active site because a single mRNA molecule gives rise to multiple protein copies. Thus, the selective inhibition of expression of a gene product required for cellular function yields the elusive but highly desired goal of chemotherapy: selective cell death. Such approaches are known in the literature, e.g., see J. S. Cohen, “Antisense oligonucleotides as an Approach Toward Anti-Aids Therapy” at pages 195-224 in Design of Anti-Aids Drugs, E. deClerg (Ed), Elsevier Publishing Co. (1990); and S. L. Loke, et al. Current Topics in Microbiology and Immunology 141: 282-289 (1988).

For enternal applications, particularly suitable are tablets, dragees, liquids, drops, suppositories, or capsules, A syrup, elixir, or the like can be used wherein a sweetened vehicle is employed. Sustained or directed release compositions can be formulated, e.g., liposomes or those wherein the active virus is protected with differentially degradable coatings, e.g., by microencapsulation, multiple coatings, etc. It is also possible to freeze-dry the compositions and use the lyophilizates obtained.

Generally, the preparations of this invention are dispensed in unit dosage form comprising 10⁶-10¹⁴ pfu/ml of the replication-deficient adenovirus in a pharmaceutically acceptable carrier per unit dosage, preferably about 10¹⁰-10¹² pfu/ml. The dosages of the biologically active compounds administered according to this invention are generally known in the art but will frequently be reduced because of the improved delivery system provided by the present invention.

The actual preferred amounts of replication-deficient adenovirus administered in a specific case will vary according with the specific protein or polypeptide being utilized, the particular compositions formulated, the mode of application, and the particular situs and organism being treated.

The particular formulation employed will be selected according to conventional knowledge depending on the properties of the protein or polypeptide and the desired site of action to ensure bioavailability of the active ingredients, i.e., the extent to which the drug reaches its site of action or a biological fluid from which the drug has access to its site of action. Dosages for a given host can be determined using conventional considerations, e.g., by customary comparison of the differential activities of the subject preparations and a known appropriate, conventional pharmacological protocol.

The present invention is described in further detail in the following non-limiting examples.

### EXAMPLES

To demonstrate the feasibility of the invention, the replication deficient recombinant adenovirus Ad-α1AT (FIG. 1) was used to transfer the sequences coding for human α1-antitrypsin to the epithelial cells of the colon. Three models were used: (1) T84 human colon carcinoma cells in vitro; (2) intact rat colon ex vivo; and (3) cotton rat colon in vivo.

The following protocols and experimental details are referenced in the Examples that follow:

The recombinant vector (Ad-α1AT) is constructed by deleting the majority of the E3 region and 2.6 mu from the left end of Ad5, and adding to the left end the α1-antitrypsin (α1AT) expression cassette from the plasmid pMLP-α1AT, containing regulatory sequences and a recombinant human α1AT gene (FIG. 1). Ad% is commercially available from the American Type Culture Collection, Rockville, Md., U.S.A. The methods for generating the α₁AT cDNA, the expression cassette, and the final vector adenovirus are prepared using the methods described by M. Rosenfeld, et al. in Science 252: 431-434 (1991).

The bottom of FIG. 1 presents details of the α1AT expression cassette. ITR, inverted terminal repeat. To construct the recombinant viral vector Ad-α1AT, the expression cassette was ligated with ClaI precut Ad-d1327 DNA (to remove a portion of the E1a region from Ad-d1327). The recombinant adenovirus DNA was transfected into the 293 cell line where it was replicated, encapsulated into an infectious virus, and isolated by plaque purification. Individual plaques were amplified by propagation in 293 cells and viral DNA extracted. The intactness of the DNA of the recombinant virus was confirmed prior to use by restriction fragment analysis and Southern hybridization. Stocks of Ad-α1AT were propagated and titered in 293 cells. The virus was released from infected cells 36 hours post-infection by 5 cycles of freeze/thawing. The Ad-α1AT was further purified using CsCl gradients (for further details see Rosenfeld M et al. (1991) Science 252:431-434).

Figure three presents a demonstration of de novo synthesis and secretion of human α1-antitrypsin (α1AT) by rat colon exposed to the recombinant adenovirus Ad-α1AT. The colon was washed, the end tied off to make a “sausage” 2-5 cm. in length, and 50-100 microliters of 10¹⁰-10¹² pfu/ml Ad-alpha1AT in LHC-8 medium injected into the lumen. The “sausage” was then incubated for 24 hr. 37°, washed, cut into 1mm² fragments, and ³²S-methionine (500 mCi/ml) added in methionine-minus LHC-8 medium. After incubation for 24 hr, 37°, the fluid bathing the fragments were evaluated for the presence of human α1AT by immunoprecipitation, sodium dodecyl sulfate acrylamide gels and autoradiography (see reference 2 for methods). Lane 1—uninfected colon; lane 2—colon infected with Ad-α1AT; and lane 3—same as lane 2, but with the antibody exposed to unlabeled human α1AT (to demonstrate the specificity of the antibody). The 52 kDa human α1AT is indicated by the arrow.

### Example 1

### T84 Human Colon Carcinoma Cells In Vitro

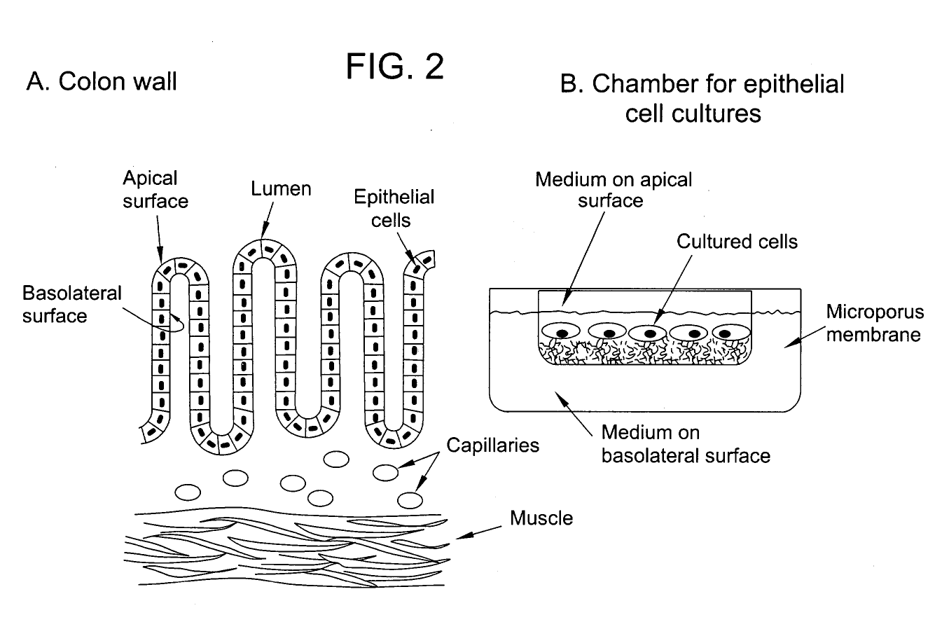

This model was used to demonstrate that human colon epithelial cells can be infected by Ad-α1AT, and that the infection resulted in the secretion of human α1AT to the apical surface (i.e., to the lumen side of the epithelium) and to the basolateral surface (i.e., the circulation side of the epithelium). To accomplish this, the T84 cell line was grown on microporous membranes until they became confluenent and formed tight junctions (electrical resistance >150 ohm-cm² across the epithelium). The microporous polycarbonate membrane (4.7 cm², pore size 3.0 um, Transwell Col., Coster, Cambridge, Mass.) with the epithelial cells separate two chambers that contain culture fluid, i.e., an in vitro system that mimics the epithelium in vivo. The upper chamber faces the apical surface and the lower chamber faces the basolateral surface. The combination of the cells and the tight junctions between the cells physically separate the fluids and the upper and lower chambers (equivalent to the in vivo situation where the apical surface abuts the inside lumen of the colon and the basolateral surface abuts the tissue side (and thus the circulation; see FIG. 2). The cells are cultured in DMEM, 2% fetal calf serum for 1.5 hr, 37° and then in DMEM, 10% fetal calt serum for 24 hr, 37° with no adenovirus or with Ad-α1AT (from the apical side as would occur in vivo). Three different intensities of infection were used [measured in plaque-forming units (pfu), the number of infectious viral particles per ml of fluid; 5×10⁹, 10¹⁰, and 2.5×10¹⁰ pfu/culture]. The media was then collected and evaluated for the presence of human α1-antitrypsin using an enzyme-linked immunoassay (Wewers M D et al. (1987) N Engl J Med 316: 1055-1062). The data demonstrates that Ad-α1AT infection causes the human colon epithelial cells to secrete α1AT, and to do so in both directions, i.e., to the apical and basolateral surfaces. The amount secreted to the apical surface compared to the basolateral surface ranged from 3.98 to 4.69 (average 4.34) i.e., for every 4.34 molecules secreted into the lumen (where it would eventually be excreted in vivo), 1 molecule would be secreted into the tissue (where it would be available to the circulation).

The results of Example 1 are shown in Table 1.

TABLE I
Polarity of Secretion of Human αl-Antitrypsin
by the Human T84 Colon Carcinoma
Cell Line Following Infection by the
Recombinant Adenovirus Ad-α1AT

| | | | Ratio of α1AT |
|---|---|---|---|
| | Amount α1AT Secreted | | in Apical |
| | in 24 hr (μg)² | | and Basolateral |
| Infection¹ | Apical | Basolateral | Compartments |
| None | 0 | 0 | — |
| 5 × 10⁹ | 3.54 | 0.89 | 3.96 |
| 10¹⁰ | 7.42 | 1.58 | 4.69 |
| 2.5 × 10¹⁰ | 8.89 | 2.05 | 4.34 |

¹number of pfu Ad-α1AT added to the culture; all cells were grown on 4.7 cm² microporous membranes until tight junctions were formed (electrical resistance > 150 ohm-cm²)
²Measured by enzyme linked immunoassay (Wewers MD et al. (1987) N Engl J Med 316: 1055-1062)

### Example 2

### Intact Rat and Cotton Rat Colon In Vitro

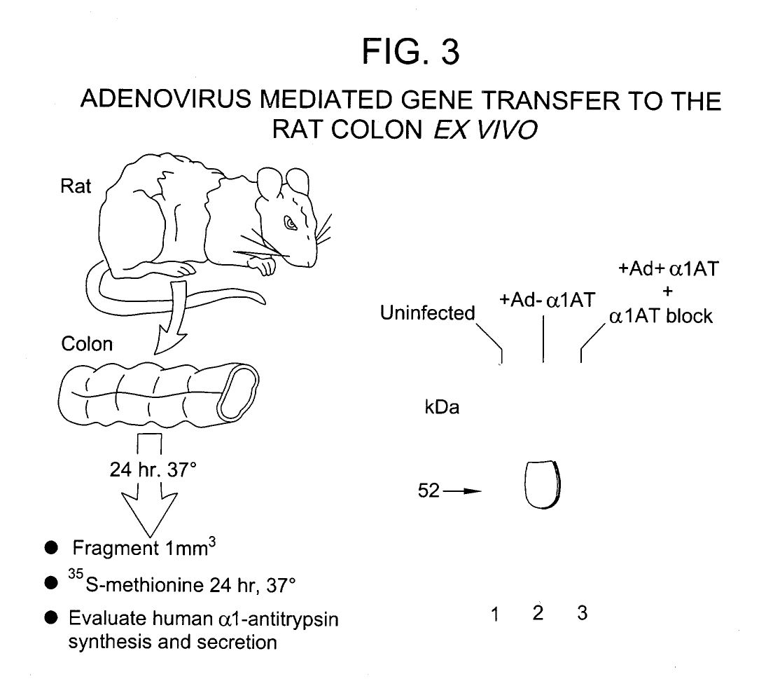

This model was used to determine if the recombinant adenovirus can infect colon epithelial cells in circumstance where the cells were normal (i.e., not derived from a neoplasm as in the T84 model) and were in their normal architectural configuration. To do this, rat colon was removed, washed and a 2-3 cm section made into a closed “sausage” by tying off both ends (FIG. 3). Ad-α1AT was injected into the lumen (e.g., equivalent to live recombinant adenovirus being released from enteric coated capsules). The “sausage” was placed in culture media for 24 hr, 37° and then evaluated in two ways.

First, the colon was fragmented into 1 mm³ pieces, ³⁵S-methionine was added, the culture continued for 24 hr, 37°, and the ability of the colon to de novo synthesize and secrete human α1AT evaluated using immunoprecipitation, sodium dodecyl sulfate acrylamide gels and autoradiography (see Rosenfeld M et al. (1991) Science 252:431-434 for details of the methods). The results demonstrate that uninfected rat colon does not synthesize and secrete human α1AT in vitro, but that Ad-α1AT infected rat colon does (FIG. 3).

A similar technique was used to evaluate cotton rat colon, but using enzyme-linked immunoassay (ELISA) to quantify the amount of human α1AT secreted into the lumen (i.e., apical secretion; it is not possible to evaluate basolateral secretion in this model). Following 48 hr infection with approximately 10¹¹ pfu Ad-α1AT injected into the lumen of cotton rat colon “sausage” in vitro, evaluation of the luminal fluid demonstrated 3.31 0.6 μg/ml human α1AT.

### Example 3

### Cotton Rat Colon In Vivo

This model was used to demonstrate that the concept will work in vivo in living animals. Two strategies were used, both in cotton rats. First, following general anesthesia and laparotomy, a section of colon was ligated in two places to form an in vivo “sausage” in a fashion that permitted normal blood flow to that segment. Ad-α1AT was injected into the lumen and the laparotomy closed. The animals were maintained without oral intake. After 48 hr, a serum sample was taken and evaluated for the presence of human α1AT by ELISA. Second, following general anesthesia and laparotomy 10¹⁰-10¹² pfu of, Ad-α1AT was injected into the lumen of the colon without ligation. After 48 hr, a serum sample was taken and evaluated for human α1AT by ELISA. In both cases human α1AT was clearly evident.

The results are shown in Table II.

TABLE II
Serum Levels of Human α1-antitrypsin in Cotton
Rats 48 hr Following In Vivo Administration of Ad-
α1AT in the Lumen of the Colon

| | Serum α1AT |
|---|---|
| Condition | Level (ng/ml)³ |
| Mock infection | 0 |
| “Sausage” infection¹ | 145 ± 29 |
| Direct infection² | 74 ± 9 |

¹Ad-α1AT (50-100 μl, approximately 10″ pfu) injected into the lumen of a segment of bowel isolated by ligation at both ends
²Similar to the “sausage” infection, but without isolating a segment of bowel by ligation
³Measured by enzyme linked immunoassay (Wewers MD et al. (1987) N Engl J Med 316: 1055-1062)

While the foregoing invention has been described in some detail for purposes of clarity and understanding, it will be appreciated by one skilled in the art from a reading of this disclosure that various changes in form and detail can be made without departing from the true scope of the invention and appended claims.

What is claimed is:

1. A method of producing a biologically active protein in the gastrointestinal tract of an individual, comprising:
administering to said individual's gastrointestinal tract a replication deficient adenovirus comprising a DNA segment encoding said protein in an amount effective and under conditions such that said protein is produced.

2. The method according to claim 1, wherein said protein is a therapeutic protein.

3. The method according to claim 1, wherein said protein is selected from the group consisting of a coagulation factor, a pituitary hormone, a peptide hormone, a lymphokine, a cytokine, a tumor supressor protein, a hematologic growth factor, a receptor agonist, and a receptor antagonist.

4. The method according to claim 1, wherein said protein is selected from the group consisting of α1-antitrypsin, erythropoietin, Factor VIII, growth hormone, tumor necrosis binding protein, interleukin-1 receptor antagonist, interferon γ, interferon α, and insulin.

5. The method according to claim 1, wherein said adenovirus is Ad-α1AT.

6. The method according to claim 1, wherein said adenovirus is administered in an enteric capsule.

7. An enteric capsule comprising a replication deficient adenovirus containing a DNA segment encoding a therapeutic protein.

8. A method of producing a biologically active protein in the gastrointestinal tract of an animal, comprising:
administering to said mammal's or bird's gastrointestinal tract a replication deficient adenovirus comprising a DNA segment encoding said protein in an amount effective and under conditions such that said protein is produced.

9. The method according to claim 8, wherein said animal is a mammal, avian or fish.

10. The method according to claim 9, wherein said mammal is selected from the group consisting of pig, sheep, cattle, horse, cat, and dog.

11. The method according to claim 9, wherein said bird is a chicken.

12. A pharmaceutical composition comprising:
a replication deficient adenovirus containing at least one DNA segment encoding for a therapeutic protein, said adenovirus contained in a vehicle that is acid-resistant and base-sensitive and
a pharmaceutically acceptable diluent, carrier, or excipient.

13. A method of developing immunity against a protein in an animal comprising:
administering to said animal's gastrointestinal tract a replication deficient adenovirus comprising a DNA segment encoding said protein in an amount effective and under conditions such that said protein is produced and said immunity against said protein is developed.
